(19) United States
(12) Patent Application Publication
(10) Pub. No.: US 2011/0004009 A1
(43) Pub. Date: Jan. 6, 2011

(54) GAS-LIQUID DISPERSION DEVICE AND METHOD FOR PRODUCING GAS-LIQUID DISPERSION

(75) Inventors: Kenji Itoh, Ichihara-shi (JP); Mayuko Yamashita, Ichihara-shi (JP); Shinjiro Ishihara, Ichihara-shi (JP)

Correspondence Address: SUGHRUE MION, PLLC, 2100 PENNSYLVANIA AVENUE, N.W., SUITE 800, WASHINGTON, DC, 20037, US

(73) Assignee: Sumitomo Chemical Company, Limited, Chuo-ku (JP)

(21) Appl. No.: 12/920,780

(22) PCT Filed: Feb. 25, 2009
(86) PCT No.: PCT/JP2009/054001

§ 371 Date: Sep. 2, 2010

(30) Foreign Application Priority Data
Mar. 4, 2008 (JP) 2008-053041

(51) Int. Cl. C07D 301/02; B01F 5/06; B01F 3/04; B01J 8/02

(52) U.S. Cl. 549/518; 261/113; 261/94; 422/211

(57) ABSTRACT

The present invention relates to a gas-liquid dispersion device which is used in a column wherein a gas-liquid mixed fluid flows upward which comprises a liquid as a continuous phase and a gas as a dispersed phase, characterized in that the gas is dispersed effectively into the liquid, so that a sufficient contact between the gas and the liquid can be attained. The device is characterized in that (A) the plate has at least one hole through which the gas and the liquid pass, (B) one end of the conduit is connected to the hole at a lower surface of the plate so that the conduit extends downward from the plate, (C) at least one passage for the gas is provided through a side surface of the conduit, and (D) at least one passage for the liquid is provided in a lower part of the conduit.

### TECHNICAL FIELD

The present invention relates to a device and a method for producing a gas-liquid dispersion. Particularly, the present invention relates to a gas-liquid dispersion device which is used in a column wherein a gas-liquid mixed fluid flows upward which comprises a liquid as a continuous phase and a gas as a dispersed phase, characterized in that the gas is dispersed effectively into the liquid, so that a sufficient contact between the gas and the liquid can be attained, and furthermore the production of the device is comparatively easy. Also, the present invention relates to a method for producing the gas-liquid dispersion characterized by using such device.

### BACKGROUND ART

In chemical industries, a so-called gas-liquid dispersion device is often utilized for dispersing a gas into a liquid in a container and thereby effective contact between the gas and the liquid is attained. A device for reacting a gas with a liquid and a device for absorbing a gas into a liquid are exemplified. In general, these devices have such a structure as blowing the gas through a bottom of a column filled with the liquid. In this case, an effective dispersion of the gas into the liquid is necessary.

In Patent Literature 1 referred to below, there is described a device for contacting a gas with a liquid in a column wherein a gas-liquid mixed fluid flows upward which comprises the liquid as a continuous phase and the gas as a dispersed phase.

However, considering that a high level efficiency is required for the dispersion, a further effective dispersion is required. From an industrial viewpoint, easy production of a device for gas-liquid dispersion is also required.

Patent Reference 1: Japanese Patent (Unexamined) Publication No. 10-118473

### DISCLOSURE OF INVENTION

Under the situation mentioned above, the problems to be solved by the present invention are providing a gas-liquid dispersion device and a method for producing the gas-liquid dispersion utilizing the device in a column wherein a gas-liquid mixed fluid flows upward which comprises a liquid as a continuous phase and a gas as a dispersed phase, characterized in that the gas is dispersed effectively into the liquid, so that a sufficient contact between the gas and the liquid can be attained, and furthermore the production of the device is comparatively easy.

In the first aspect, the present invention provides a gas-liquid dispersion device which comprises a plate for blocking a flow of a fluid and a conduit for conducting the fluid through the plate from a lower side to an upper side of the plate in a column wherein a gas-liquid mixed fluid as said fluid flows upward which comprises a liquid as a continuous phase and a gas as a dispersed phase, characterized in that:
(A) the plate has at least one hole through which the gas and the liquid pass,
(B) one end of the conduit is connected to the hole (for gas-liquid mixed fluid at a lower surface of the plate so that the conduit extends downward from the plate,
(C) at least one passage for the gas is provided through a side surface of the conduit,
(D) at least one passage for the liquid is provided in a lower part of the conduit, and
(E) an end of the lower part of the conduit has a structure for preventing the gas from flowing into the conduit.
   (It is noted that a part of the conduit including the other end of the conduit which is not connected to the hole for gas-liquid mixed fluid is referred to as a “lower part”)

In the second aspect, the present invention provides a method for producing a gas-liquid dispersion of a gas-liquid mixed fluid, which method comprises the steps of:
i) flowing the gas-liquid mixed fluid upward through a column comprising the gas-liquid dispersion device according to the present invention wherein the gas-liquid mixed fluid is formed of a continuous phase of the liquid and a dispersed phase of the gas;
ii) forming a space (which may be referred to as a gas-cumulating chamber) comprising the gas under the plate;
iii) conducting the liquid into the conduit through the passage for the liquid provided to the conduit;
iv) conducting the gas into the conduit through the passage for the gas provided to the conduit;
v) forming the gas-liquid mixed fluid by mixing the liquid and the gas in the conduit; and
vi) then, flowing the gas-liquid mixed fluid upward through the hole for gas-liquid mixed fluid which the plate has.

In the third aspect, the present invention provides a method for producing the gas-liquid dispersion mentioned above, wherein the method is utilized in a hydrogenation step in a propylene oxide production process comprising:
i) an oxidation step for obtaining an aklylbenzene hydoroperoxide by oxidizing an alkylbenzene;
ii) an epoxidation step for obtaining a reaction liquid containing propylene oxide and an alcohol derived from the alkylbenzene hydroperoxide by reacting the alkylbenzene hydroperoxide and propylene in the presence of a catalyst;
iii) a propylene recovery step for recovering unreacted propylene from the reaction liquid after the epoxidation step, and for recycling the recovered propylene to the epoxidation step as a raw material;
iv) a propylene oxide purification step for obtaining purified propylene oxide by, for example, distilling the propylene oxide obtained by the epoxidation step; and
v) the hydrogenation step for obtaining the alkylbenzene by hydrogenation, in the presence of a catayst, of the alcohol obtained from the alkylbenzene hydroperoxide via the epoxidation step, and for recycling the alkylbenzene as a raw material to the oxidation step.

### BRIEF DESCRIPTION OF DRAWINGS

FIG. 1 is a schematic view of an apparatus used in Example 1, which comprises the gas-liquid dispersion device according to the present invention.

FIG. 2 is a schematic view of an apparatus used in Example 2, which comprises the gas-liquid dispersion device according to the present invention.

FIG. 3 is a schematic flow chart of Example 3.

FIG. 4 is a schematic view of the conduit of the device according to the present invention wherein the end of the lower part of the conduit has a structure which is closed by a cap.

FIG. 5 is a schematic view of the conduit of the device according to the present invention wherein the lower part of the conduit has a structure which is bent in the form of “J” character.

FIG. 6 is a schematic view of the conduit of the device according to the present invention wherein two or more of the passages for the gas per one conduit are provided through the conduit and the passages for the gas are positioned at two or more different levels of the conduit.

FIG. 7 is a schematic view of the conduit of the device according to the present invention wherein the passage for the gas is in the form of a slit.

### DESCRIPTION OF REFERENCE NUMERALS

1: Column
2: Liquid
3: Gas
4: Plate
5: Hole for gas and liquid
6: Conduit
7: Passage for gas
8: End of lower part of conduit
9: Passage for liquid
10: Bed of catalyst packing
11: Outlet of gas and liquid
12: Hole for gas and liquid
13: Conduit
14: Passage for gas
15: End of lower part of conduit
16: Passage for liquid
17: Water
18: Air
19: Layer of air
20: Tube
21: Plate
22: Outlet of gas and liquid
101: Oxidation reaction liquid
102: Epoxidation reaction liquid
103: Unreacted propylene
104: Reaction liquid after recovering propylene
105: Liquid mainly containing cumyl alcohol and cumene
106: Recycle cumene

### EMBODIMENT FOR CARRYING OUT THE INVENTION

In accordance with the aspect of the present invention, the column is used wherein the gas-liquid mixed fluid flows upward, the liquid forms a continuous phase and the gas forms a dispersed phase. That is, the liquid and the gas are fed from the bottom of the column, the gas-liquid mixed fluid is formed wherein the gas is present as a dispersed phase in the liquid as a continuous phase, the gas-liquid mixed fluid flows up through the column, and it was withdrawn out of the column from around the top of the column. And, within the column, the gas is dispersed in the liquid, and thus a sufficient contact between the gas and the liquid is accomplished.

The present invention provides the gas-liquid dispersion device comprising the plate for blocking the flow of the fluid and the conduit for conducting the fluid through the plate from the lower side to the upper side of the plate in the column wherein the gas-liquid mixed fluid flows upward which comprises the liquid as the continuous phase and the gas as the dispersed phase, and the device satisfied the following:
(A) the plate has at least one hole through which the gas and the liquid pass,
(B) one end of the conduit is connected to the hole (for gas-liquid mixed fluid at the lower surface of the plate so that the conduit extends downward from the plate,
(C) at least one passage for the gas is provided through the side surface of the conduit,
(D) at least one passage for the liquid is provided in the lower part of the conduit, and
(E) the end of the lower part of the conduit has the structure for preventing the gas from flowing into the conduit.

According to the present invention, (A) the plate has at least one hole through which the gas and the liquid pass, (B) one end of the conduit is connected to the hole for gas-liquid mixed fluid at the lower surface of the plate, the conduit extending downward, (C) at least one passage for the gas is provided through the side surface of the conduit, (D) at least one passage for the liquid is provided in the lower part of the conduit, and (E) the end of the lower part of the conduit has the structure for preventing the gas from flowing into the conduit.

Since the end of the lower part of the conduit has the structure for preventing the gas from flowing into the conduit, only the liquid can be introduced into the conduit from the end of the lower part of the conduit. The wording “the end of the lower part of the conduit” means one of the both ends which is not connected to the hole for gas-liquid mixed fluid of the plate, and which includes the side surface around the end. Preferable examples of the end structure are the capped end type with the end being closed by the cap and the J-shaped type. The liquid can be introduced into the conduit through any one or more of the following passages: i) a small hole(s) provided through the side surface of the conduit, ii) an opening like a slit through the side surface of the conduit around the connection between the cap and the conduit, and iii) an opening through the end of the conduit being bent in the form of “J” character.

It is preferable that the diameter of the hole for gas-liquid mixed fluid is generally the same as that of the conduit. Thereby, designing and manufacturing of the device can be made easily.

In the present invention, the following equation (1) is preferably satisfied:

1≦N/S≦100 (1)

wherein N is the number of the holes for gas-liquid mixed fluid per one plate, and S is an area [m²] of the lower surface (or “upper surface”) of the plate.

If N/S is excessively small, re-dispersion may be insufficient, whereas if N/S is excessively large, the numbers of components of the device may be large and thereby both the production cost and the weight of the device may increase to cause an installation problem of the device.

The areas of the upper and the lower surfaces of the plate are generally equal.

In the present invention, the following equation (2) is preferably satisfied:

0.01≦v≦10 (2)

wherein v is a linear velocity [m/s] of the gas-liquid mixed fluid flowing through the hole for gas-liquid mixed fluid.

If v is excessively small, a pressure drop across the hole for gas-liquid mixed fluid is small and flow rates of the gas and the liquid through the plural holes tend to become uneven to result in a channeling problem, whereas if v is excessively large, the pressure drop across the hole for gas-liquid mixed fluid is large, and thereby a cost of an apparatus comprising the device becomes higher in order to strengthen an apparatus endurance and to increase delivery pressures of a pump and a compressor which supply the liquid and the gas to the apparatus.

In the present invention, the following equation (3) is preferably satisfied:

100≦1.5×L≦H (3)

wherein H is a height of the column [mm], and L is a length of the conduit [mm].

If L is excessively small, the height of the space comprising the gas under the plate which space is required to re-disperse the gas is insufficient. In such a case, the conduit may be unable to accommodate to changes of the space height caused by changes of operation conditions and throughputs. On the other hand, if L is excessively large, concern about vibration of the conduit increases, and thereby means for sufficient vibration suppression becomes necessary. In addition, since the space where the conduit is provided is generally not loaded with packing such as a catalyst, the amount of the loaded packing disadvantageously decreases.

In the present invention, the following equation (4) is preferably satisfied:

10≦g≦500 (4)

wherein g is a velocity [m/s] of the gas flowing through the passage for the gas provided through the side surface of the conduit, provided that velocity is based on the volume of the gas of the entrance side of the passage for the gas.

If g is excessively small, the space of the gas under the plate which space being necessary for re-dispersing of the gas is not formed sufficiently, so that the liquid may flow into the conduit through the passage for the gas provided through the side surface of the conduit. On the other hand, if g is excessively large, the height of the space of the gas under the plate may exceed the length of the conduit, so that the gas may flow into the conduit together with the liquid from the passage for the liquid provided through the lower part of the conduit. Thus, the efficient gas dispersion as the aim of the present invention may not be achieved.

In the present invention, the following equation (5) is preferentially satisfied:

1≦h≦10 (5)

wherein h is a velocity [m/s] of the liquid flowing through the passage for the liquid provided through the conduit, provided that the velocity is based on the actual volume of the liquid.

If h is excessively small, the height of the space of the gas may exceed the length of the conduit, so that the gas may flow into the conduit together with the liquid from the passage for the liquid provided through the lower part of the conduit. Therefore, the efficient gas dispersion as the aim of the present invention may not be achieved. On the other hand, if h is excessively large, the space of the gas can not be formed sufficiently, and thereby the liquid may flow into the conduit from the passage for the gas provided through the side surface of the conduit.

In the present invention, the following equation (6) is preferably satisfied:

−4≦dP_{G}−dP_{L} (6)

wherein dP_{G} is the pressure drop across the passage for gas [kPa], and dP_{L} is the pressure drop across the passage for liquid [kPa]. Preferably dP_{G}−dP_{L} is zero or more, or further preferably dP_{G}−dP_{L} is one or more.
Here, dP_{G} is a numerical value derived from the formula of a gaseous restriction orifice, and can be calculated in the following procedure.
(1) Under the pressure condition of Pₐ>P_{c}, the critical pressure of P_{c} is calculated from the following equation (a).

P_{c}=Pₐ×(2/(κ+1))^{(κ(κ-1))} (a)
(2) The gas flow coefficient of C_{G} is calculated from the following equation (b).

C_{G}=2.80×(κ×(2/(κ+1))^{((κ+1)/(κ-1))})^{0.5} (b)
(3) The critical orifice diameter of d_{c} is calculated by the following equation (c).

d_{c}=(W_{G}/(C_{G}×Pₐ×(M/T)^{0.5}))^{0.5} (c)
(4) The secondary pressure P_{b} with which it is satisfied of a restriction orifice equation (d) is calculated.))

d_{G}=d_{c}×((2/(κ+1))^{(2/(κ-1))}×((κ−1)/(κ−1)/((P_{b}/Pₐ)^{(2/κK)}−(P_{b}/Pₐ)^{((κ+1)/κ)}))^{0.25} (d)
(5) The pressure drop across the passage for gas is calculated by the following equation (e)

dP_{G}=(P_{b}−Pₐ)×98.07 (e)

wherein d_{G} is orifice (passage for the gas) diameter [mm], κ is Ratio of specific heat [−], Pₐ is the primary pressure [kg/cm²A], P_{b} is the secondary pressure [kg/cm²A], P_{c} is the critical pressure [kg/cm²A], W_{G} is mass flow rate [kg/hr], C_{G} is the gas flow coefficient, T is temperature [K], d_{c} is the critical orifice diameter [mm], M is molecular weight of average gas [g/mol]

On the other hand, dP_{L} is calculated by the following equation (f).

dP_{L}=(W_{L}/(39.6×C_{L}×d_{L}²))²/ρ_{L}×98.07 (f)

wherein W_{L} is mass flow rate [kg/hr], C_{L} is the liquid flow coefficient, d_{L} is orifice (passage for the liquid) diameter [mm], ρ_{L} is liquid specific gravity of liquid (water at 4 degree C., 1 atm standard) [−]

But when a passage (for the gas or the liquid) is not the orifice of the circle hole, the equivalent diameter of dₑ is calculated by the following equation (g), and it is used as d_{G} or d_{L}.

dₑ=4×sₑ/Lₑ (g)

wherein dₑ is the equivalent diameter [mm], sₑ is sectional area of a passage (for the gas or the liquid) [mm²], L_{g} is the length of wetted (touched to gas or liquid) perimeter.

If dP_{G}−dP_{L} is excessively small, the space of the gas under the plate which space being necessary for re-dispersing of the gas is not formed sufficiently, so that the liquid may flow into the conduit through the passage for the gas provided through the side surface of the conduit. On the other hand, if dP_{G}−dP_{L} is excessively large, the height of the space of the gas under the plate may exceed the length of the conduit, so that the gas may flow into the conduit together with the liquid from the passage for the liquid provided through the lower part of the conduit. Thus, the efficient gas dispersion as the aim of the present invention may not be achieved.

In the present invention, it is preferable that two or more of the passages for the gas per one conduit are provided through the conduit and such passages for the gas are positioned at two or more different levels of the conduit. A shape of the passage for the gas is not particularly restricted but it may be a circular hole having a diameter of 1 mm to 20 mm. If an opening area of one passage is excessively small, the number of the required passage increases, and thereby a manufacturing cost of the conduit increases. On the other hand, if the opening area of one passage is excessively large, an operable range of the conduit narrows. Therefore, a skilled person can determine an economically reasonable diameter. In this way, the device can flexibly accommodate the changes of the height of the space of the gas due to the changes in the operation conditions and the throughput. In particular, when the flow rate of the gas is smaller compared to that of the liquid and the flow speed of the gas through the passage for the gas is slow, the height of the space of the gas becomes lower, and thereby only the passage for the gas close to the plate can work. When the gas rate is large and the height of the space of the gas becomes higher, the passage for the gas remote from the plate can also work. Thus, even when the gas rate is small, the required height of the space of the gas can be kept, and even when the gas rate is large, the height of the space of the gas does not exceed the lower end of the conduit, so that therefore, the range where the present invention can be carried out can be broaden.

In the present invention, one preferable device comprises at least one slit-type opening per one conduit provided longitudinally through the side surface of the conduit, and at least a part of such opening functions as the passage for the gas. In the case wherein the slit is provided longitudinally through the side surface of the conduit, when the height of the space of the gas is lower, only a part of the slit-type opening which is closer to the plate can work as the passage for the gas. On the other hand, when the gas flow rate increases, and the height of the space of the gas becomes higher, a part of the slit-type opening corresponding to such height can work as the passage for the gas. When the width of the slit-type opening is too wide, no sufficient space of the gas may be formed. Therefore, the width of the slit-type opening is preferably between 1 mm to 10 mm.

In the present invention, a bed of a packing can be provided above and/or below the gas-liquid dispersion device.

The gas-liquid dispersion device of the present invention is characterized in that it can disperse the gas effectively into the liquid in the column wherein the gas-liquid mixed fluid flows upward which comprises the liquid as the continuous phase and the gas as the dispersed phase, so that it can attain a sufficient contact between the gas and the liquid. Therefore, it has a wide application range.

The method for utilizing the device of the present invention has been made clear from the above explanation. In short, the method for producing the gas-liquid dispersion comprising the gas as the dispersed phase and the liquid as the continuous phase comprises the steps of:
i) flowing the mixed fluid of the gas and the liquid upward through the column comprising the gas-liquid dispersion device;
ii) forming the space comprising the gas under the plate;
iii) conducting the liquid into the conduit through the passage for the liquid provided in the lower part of the conduit;
iv) conducting the gas into the conduit through the passage for the gas provided through the side surface of the conduit;
v) forming the gas-liquid mixed fluid by mixing the liquid and the gas in the conduit;
vi) then, flowing the gas-liquid mixed fluid upward through the hole for gas-liquid mixed fluid provided through the plate.

Applications of the present invention include gas-liquid contact in a packed column, gas-liquid contact in a bubble column and the like. In more specific example, the packing is a catalyst and especially the catalyst utilized in hydrogenation or dehydration.

According to the present invention, the liquid and the gas are mixed in the conduit, and the mixed fluid is shed upward through the hole for gas-liquid mixed fluid of the plate, and thereby an efficient contact between the gas and the liquid can be achieved. And, as can be seen from Example 1 and Comparative Example 1 which will be described bellow, because of the sufficient contact between the gas and the liquid, the catalytic gas-liquid reaction can be carried out in an excellent performance. Furthermore, the gas-liquid dispersion device of the present invention requires only one kind of the hole for gas-liquid mixed fluid provided through the plate, and therefore, it has an advantage that the fabrication of the device upon its production is comparatively easy.

As the best way to carry out the present invention from the industrial viewpoint, a gas-liquid dispersion method can be exemplified which is used in a hydrogenation step in the production of propylene oxide comprising:
i) an oxidation step for obtaining an aklylbenzene hydoroperoxide by oxidizing an alkylbenzene;
ii) an epoxidation step for obtaining a reaction liquid containing propylene oxide and an alcohol derived from the alkylbenzene hydroperoxide by reacting the alkylbenzene hydroperoxide and propylene in the presence of a catalyst;
iii) a propylene recovery step for recovering unreacted propylene from the liquid after the epoxidation step, and for recycling the recovered propylene to the epoxidation step as a raw material;
iv) a propylene oxide rough separation step for separating crude propylene oxide from an alcohol originated from an alkylbenzene hydroperoxide obtained in the epoxidation step;
v) a propylene oxide purification step for obtaining purified propylene oxide by, for example, distilling the propylene oxide after the propylene oxide rough separation step; and
vi) a hydrogenation step for obtaining the alkylbenzene by hydrogenation, in the presence of a catalyst, of the alcohol obtained from the alkylbenzene hydroperoxide which alcohol is separated by the propylene oxide rough separation step for recycling the alkylbenzene as a raw material to the oxidation step.

The oxidation step in the present invention is a step of oxidizing the alkylbenzene to obtain the alkylbenzene hydroperoxide. Usually, the alkylbenzene is automatically oxidized using an oxygen-containing gas such as air or oxygen concentrated air. This oxidation reaction may be carried out without using an additive, or carried out using an additive such as an alkali. The reaction temperature is usually from 50° C. to 200° C., and the reaction pressure is from an atmospheric pressure to 5 MPa. In the case of the oxidation method using the additive, an alkali reagent to be used is an alkali metal compound such as NaOH or KOH, an alkali earth metal oxide, an alkali metal carbonate such as Na₂CO₃, NaHCO₃, ammonia, (NH₄)₃CO₃, or an alkali metal ammonium carbonate salt.

The epoxidation step of the present invention is a step of reacting the alkylbenzene hydroperoxide with propylene in the presence of the catalyst to obtain propylene oxide and the alcohol derived from the alkylbenzene hydroperoxide.

From a viewpoint of obtaining an aimed product at a high yield and a high selectivity, the epoxidation step is preferably carried out in the presence of a catalyst composed of a titanium-containing silicon oxide. This catalyst is usually a solid catalyst, and is preferably a so-called Ti-silica catalyst, Ti of which is chemically bonded with a silicon oxide. The catalyst includes, for example, a catalyst in which a Ti compound is supported on a silica carrier, a catalyst in which a Ti compound is combined to a silicon oxide prepared by, for example, using a co-precipitation method or a sol gel method, or a catalyst made of a zeolite compound which contains Ti.

In the present invention, the alkylbenzene hydroperoxide used as the raw material of the epoxidation step may be a diluted or concentrated purified compound or non-purified compound.

The epoxidation reaction is carried out by bringing propylene and the alkylbenzene hydroperoxide into contact with the catalyst. The reaction is carried out in a liquid phase using a solvent. The solvent must be a liquid under a temperature and a pressure upon the reaction, and must be substantially inactive with the reactants and the products. The solvent may be made of a material which exists in an alkylbenzene hydroperoxide solution to be used. For example, when ethylbenzene hydroperoxide or cumene hydroperoxide is a mixture containing ethylbenzene or cumene as a raw material thereof, it is possible to use the mixture in place of the solvent without adding a solvent. In addition, a useful solvent includes an aromatic monocyclic compound (for example, benzene, toluene, chlorobenzene, or o-dichlorobenzene), and an alkane (octane, decane, or dodecane).

The epoxidation reaction temperature is generally from 0° C. to 200° C., and is preferably from 25° C. to 200° C. in view of the reaction rate and the economical use of the catalyst, and more preferably from 25° C. to 140° C. in view of the reaction selectivity. When the temperature is too low, the reaction rate is low, and thus an amount of the catalyst, which is required to achieve a desired reaction extent, increases. In contrast, when the temperature is too high, the selectivity decreases. Particularly when the amount of the produced compound having 4 carbon atoms increases, both a loss of a valuable component and a required energy upon the removal of the compound un-advantageously increase. The pressure may be a sufficient pressure to keep a reaction mixture in a liquid state. The pressure is advantageously from 100 kPa to 10,000 kPa.

The solid catalyst is advantageously used in the form of a slurry or a fixed bed. In the case of a large-scale industrial operation, the fixed bed is preferably used. Also, the operation can be carried out by a batch method, a semi-continuous method, or a continuous method. When a liquid containing reaction raw materials is passed through the fixed bed, a liquid mixture from the reacted region does not contain any catalyst, or contains substantially no catalyst.

A molar ratio of propylene/alkylbenzene hydroperoxide to be supplied to the epoxidation step is preferably from 2/1 to 50/1. When the ratio is too low, the reaction rate decreases, and thus the reaction efficiency becomes worse. When the ratio is too high, the amount of propylene to be recycled becomes excessively increases, and thus much energy is required in the recovery step.

The propylene recovery step in the present invention is a step of separating and recovering the unreacted propylene in the reaction liquid after the epoxidation step and recycling the recovered propylene to the epoxidation step as a raw material thereof. As described above, since propylene is excessively used, the reaction liquid from the epoxidation step contains the unreacted propylene. The method of separating and recovering the unreacted propylene from the reaction liquid includes a method of distilling the reaction liquid. The reaction liquid is distilled under the conditions which enable easy evaporation of propylene from the reaction liquid. The conditions of the distillation vary depending on the temperature and the composition of the reaction liquid supplied to the distillation step. Usually, the pressure is from 100 kPa to 5,000 kPa, and preferably from 100 kPa to 3,000 kPa, and a column top temperature is from −50° C. to 150° C. A method of stepwisely distilling propylene using a plurality of distillation columns can also be used.

The propylene oxide purification step of the present invention is a step of subjecting the propylene oxide produced in the epoxidation step to, for example, distillation to obtain purified propylene oxide.

Propylene oxide to be purified is a liquid which remains after recovering the unreacted propylene from the reaction liquid of the epoxidation step as described above.

Usually, the solvent and the alcohol produced in the epoxidation step are removed first by distillation to obtain a crude propylene oxide.

The crude propylene oxide generally contains water, a hydrocarbon, and an oxygen-containing compound as impurities, and the hydrocarbon includes a hydrocarbon having, for example, 3 to 7 carbon atoms. Examples of the oxygen-containing compound include, for example, methanol, acetaldehyde, acetone, propionaldehyde, and methyl formate.

As the method of removing these impurities, for example, known separation techniques such as distillation, extraction, adsorption, and crystallization may be appropriately used in combination. However, the crude propylene oxide is preferably purified by using extraction distillation with a hydrocarbon having 7 to 10 carbon atoms as an extractant in combination with the other distillation in view of efficiently removing water, the hydrocarbon, and the oxygen-containing compound.

Examples of the hydrocarbon having 7 to 10 carbon atoms as the extractant include linear saturated hydrocarbons such as n-heptane, n-octane, n-nonane, and n-decane; branched saturated hydrocarbons such as 2,2-dimethylpentane, 2,3-dimethylpentane, 2,2-dimethylhexane, and 2,3-dimethylhexane; and unsaturated hydrocarbons thereof. In addition, these extractants can be used alone, or a mixture thereof can be used.

The type and operation conditions of the extraction distillation column and other distillation column, operation conditions, and the amount of the extractant are appropriately determined according to the qualities of the product to be required.

The purified propylene oxide thus obtained satisfies desired product qualities.

The hydrogenation step in the present invention is a step of hydrocracking or dehydrating/hydrogenating the alcohol which is derived from the alkylbenzene hydroperoxide and obtained in the epoxidation step in the presence of the solid catalyst to obtain the alkylbenzene, and recycling the resulted alkylbenzene to the oxidation step as a raw material of the oxidation step. In order to efficiently recycle the alkylbenzene, this step is preferably carried out by dehydration/hydrogenation.

The dehydration step is a step of supplying the alcohol which is derived from the alkylbenzene hydroperoxide and obtained in the epoxidation step to a dehydration catalyst to dehydrate intra-molecularly. Examples of the catalyst to be used include acids such as sulfuric acid, phosphoric acid, and p-toluenesulfonic acid; and metal oxides such as activated alumina, titania, zirconia, silica-alumina, and zeolite, of which activated alumina is preferable in view of separation from a reaction liquid, catalyst life, and selectivity.

The amount of the dehydration catalyst is an enough amount to convert the alcohol. The conversion of the alcohol is preferably 90% or more, and more preferably 98% or more.

The dehydration reaction is carried out by bringing a solution containing the alcohol into contact with the catalyst. However, in the dehydration/hydrogenation method, the hydrogenation reaction is carried out after the dehydration reaction, and thus hydrogen can be fed to the catalyst. The dehydration reaction temperature is generally from 50° C. to 450° C., and more preferably from 150° C. to 300° C. The pressure is advantageously from 10 kPa to 10,000 kPa.

The hydrogenation step is a step of supplying the intra-molecularly dehydrated product obtained in the dehydration step to the hydrogenation catalyst, hydrogenating it thereby converting into the alkylbenzene, and recycling it to the oxidation step as a raw material of the oxidation step.

The hydrogenation catalyst is a catalyst containing a metal of the group 10 or 11 in the Periodic Table, and specific examples thereof include nickel, palladium, platinum and copper, of which palladium or copper is preferable in view of suppression of the hydrogenation reaction of an aromatic ring and high yield. Examples of a copper-based catalyst include copper, Raney copper, copper-chromium, copper-zinc, copper-chromium-zinc, copper-silica, and copper-alumina. Examples of a palladium catalyst include, for example, palladium-alumina, palladium-silica, and palladium-carbon. These catalysts may be used alone, or a plurality of them may be used in combination. When the hydrogenation catalyst has also dehydration ability, the catalyst can be used as the dehydration/hydrogenation catalyst.

The amount of the hydrogenation catalyst is an enough amount to convert the intra-molecularly dehydrated product, and the conversion is preferably 98% or more.

The hydrogenation reaction is carried out by bringing the solution containing the intra-molecularly dehydrated product and hydrogen into contact with the catalyst. However, in the dehydration/hydrogenation method, the hydrogenation reaction is carried out after the dehydration reaction. Therefore, the hydrogenation reaction may be carried out after separating water produced in the dehydration reaction through, for example, oil-water separation, or may be carried out by supplying water together with the intra-molecularly dehydrated product to the hydrogenation catalyst without separating water.

The amount of hydrogen required to the reaction may be a molar amount which is equivalent to that of the intra-molecularly dehydrated product. The raw material usually contains other components which consume hydrogen, and thus an excessive amount of hydrogen is required. As a partial pressure of hydrogen increases, the reaction proceeds more quickly, and thus a mol ratio of hydrogen/intra-molecularly dehydrated product is preferably from 1 to 10, and more preferably from 1 to 5. The excessive hydrogen which remains after the reaction can be recycled after separating from the reaction liquid.

The hydrogenation reaction temperature is generally from 0° C. to 500° C., and more preferably 30° C. to 300° C. The pressure is advantageously from 100 kPa to 10,000 kPa.

The dehydration/hydrogenation reaction can be advantageously carried out using a fixed bed. The dehydration reaction and the hydrogenation reaction may be carried out using separate reactors, or may be carried out using a single reactor. It is preferable that the dehydration catalyst and the hydrogenation catalyst are not filled in multi-staged reactors, but are filled in a single fixed bed reactor in view of the cost.

Therefore, when the present invention is to be applied to the hydrogenation step in the above mentioned process for the production of propylene oxide, the gas-liquid dispersion method of the present invention can be adopted in the fixed bed reactor wherein the gas-liquid mixed fluid flows upward which fluid comprises the alcohol derived in the epoxidation step from the alkylbenzene hydroperoxide as the continuous phase and the hydrogen gas as the dispersed phase.

### Examples

The present invention is explained with reference to the Examples.

### Example 1

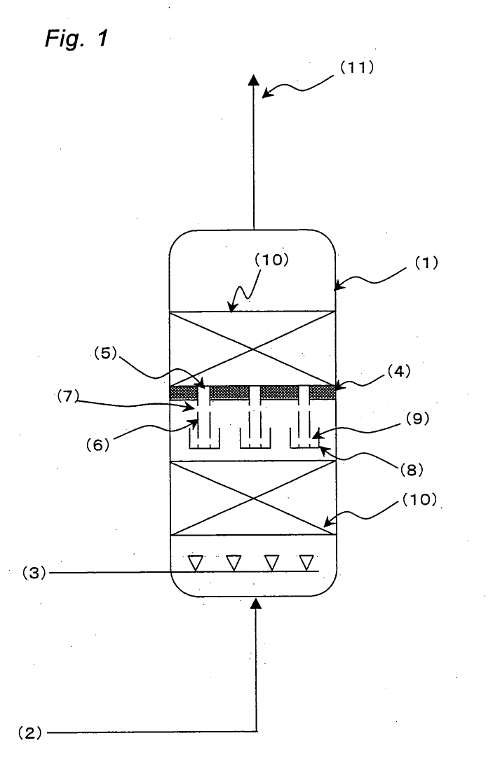

Using the apparatus as shown in FIG. 1, Example 1 was carried out. A liquid (2) (a cumene solution containing about 25% by weight of cumyl alcohol) and a gas (3) (hydrogen) were fed through the bottom of the column (1), and the resulting mixed fluid of the gas and the liquid was flowed upward through the column. Within the column, there was provided the gas-liquid dispersing device according to the present invention comprising the plate (4) which was arranged perpendicular to the flow direction of the fluid and blocked the flow of the fluid. The plate had holes (5) for gas-liquid mixed fluid, the holes were connected to the conduits (6) extending downward from the plate, and three passages (7) for the gas per one conduit were provided through the side surface of the conduit. As was described above, in order that the positional relationship in the levels of the passages for the gas through the conduit flexibly accommodates the change in the height of the space of the gas in accordance with the change of the operation conditions and the throughput, the first passage for the gas was provided at the level of 75 mm downward from the plate, the second one was at the level of additionally 40 mm downward from the first passage and the third one was at the level of further additionally 40 mm downward from the second passage. The ratio of H/L was about 12.7, wherein height of the column (1) was H and length of conduits (6) was L.

The structure of the end (8) of the lower part of the conduit had a structure which was closed by a cap. And, two small holes as the passages for the liquid per one conduit were provided through the conduit at the same level of 45 mm upward from the lower end of the conduit.

The diameter of the hole of the plate was generally the same as that of the conduit.

The ratio of N/S was 15/m² wherein N is the number of the holes of the plate and S is an area [m²] of the lower surface of the plate.

The linear velocity (v) of the gas-liquid mixed fluid flowing through the hole of the plate was about 2 m/s which is based on the volume of the fluid of the entrance (lower) side of the hole for the gas-liquid mixed fluid.

The velocity (g) of the gas flowing through the passage for the gas provided through the side surface of the conduit was about 54 m/s which is based on the volume of the gas of the entrance side of the passage for the gas.

The velocity (h) of the liquid flowing through the passage for the liquid provided through the conduit was about 6 m/s.

Thus, it was confirmed that a space (with a thickness of 380 mm) containing the gas was formed by flowing the liquid and the gas at a predetermined ratio through the conduit from the down side to the upper side of the plate. The value of dP_{G}−dP_{L} was 8.7 kPa at this point.

Above the gas-liquid dispersion device, there was provided a bed (10) of packing comprising spherical alumina catalysts (partially supporting a noble metal). Under the conditions of a catalyst bed temperature of about 200° C. to 230° C. and a column top pressure of about 1.5 MPaG to 2 MPaG, cumyl alcohol was intra-molecularly dehydrated to α-methylstylene, and α-methylstylene was successively converted to cumene by reacting with hydrogen. When the state of the gas-liquid dispersion is insufficient, there would be caused channeling in the reactor, and thereby an insufficient hydrogenation zone and an excessive hydrogenation zone would be partially formed. The formation of such zones is one of factors which worsen the loss of cumene in the hydrogenation step. Then, the state of the dispersion was evaluated by the following index. The index for the insufficient hydrogenation was a concentration of α-methylstylene at the outlet of the column (referred to as a leak-concentration of α-methylstylene). As a result, the obtained reaction product had the leak-concentration of α-methylstylene (the index for the insufficient hydrogenation) was 323 ppm by weight. In Table 1, the results of Example 1 are shown with “with dispersion plate”.

### Comparative Example 1

The same procedure as in Example 1 was carried out except that the gas-liquid dispersion device according to the present invention was not used. As a result, the leak concentration of α-methylstylene was 1400 ppm by weight. The results of Comparative Example 1 are shown in Table 1 with comparing with those of Example 1. In Table 1, the results of Comparative Example 1 are shown with “without dispersion plate”. Table 1 shows that, as to the conversion of cumylalcohol to cumene, the leak concentration of α-methylstylene un-hydrogenated was 4.3 times higher than that of Example 1.

From Example 1 and Comparative Example 1, it was found that the hydrogenation reaction was promoted by using the gas-liquid dispersing plate according to the present invention.

TABLE 1

| | Leak-Concentration of α- |
|---|---|
| | Methylstylene [ppm by weight] |
| with Dispersing Plate | 323 |
| (Example 1) | |
| without Dispersing Plate | 1400 |
| (Comparative Example 1) | |

### Example 2

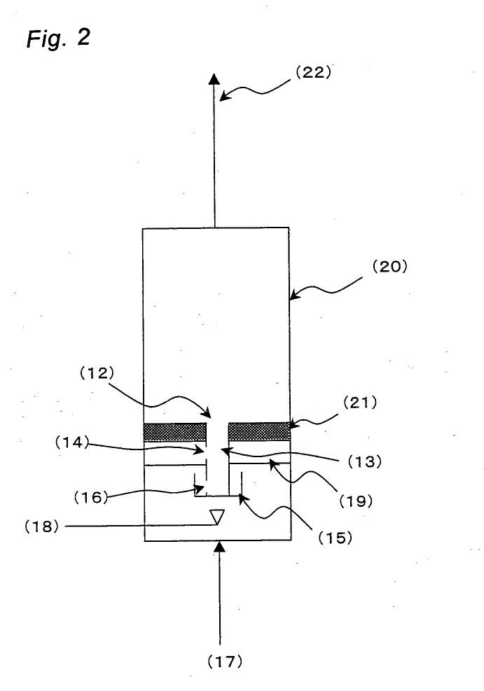

The following experiments were carried out using the apparatus as shown in FIG. 2 which comprises a tube (20) which was made of a transparent acrylic polymer (280 mm in diameter and 1980 mm in height) and was standing vertically. Within the tube is provided the gas-liquid dispersion device according to the present invention which comprises a plate (21) provided with one hole for a gas (12) (50 mm in diameter) and one conduit (13) (50 mm in diameter and 1000 mm in height), and one passage (14) for the gas (6 mm in diameter) at a level of 45 mm downward from the upper end of the conduit. The ratio of H/L was about 1.98, wherein height of the tube (20) was H and length of conduits (13) was L.

The end of the lower part of the conduit had the structure which was closed by a cap, and, inside the capped structure, one small hole (16) (10 mm in diameter) as a passage for the liquid was provided through the side surface of the conduit. The tube was filled with water, and then, water and air were fed from a lower part of the tube under the conditions described in Table 2, so that the value of dP_{G}−dP_{L} was more than −4kPa (see Table 2, experimental 1 and 2). As a result of the visual observation, it was confirmed that the thickness of a space comprising the gas under the plate described in Table 2 was formed, a mixed fluid of the gas and the liquid blew off upward from the hole of the plate, and thereby extremely good dispersion was achieved.

### Comparative Example 2

The same procedure as in Example 2 was carried out except that conditions were different from Example 2. The value of dP_{G}−dP_{L} was under −4 kPa. (See Table 2, comparative).

TABLE 2

| | | | | | | thickness of space | |
|---|---|---|---|---|---|---|---|
| | | | | | | comprising gas | |
| | N/S | v | g | h | dP_{G}-dP_{L} | under plate | |
| | —/m² | m/s | m/s | m/s | kPa | mm | dispersion |
| Experiment 1 | 16 | 1.2 | 75 | 2.1 | 1.0 | 230 | GOOD |
| Experiment 2 | 16 | 1.5 | 99 | 2.1 | 4.4 | 608 | GOOD |
| Comparative | 16 | 1.2 | 73 | 3.4 | −4.7 | Non | Bad |

### Example 3

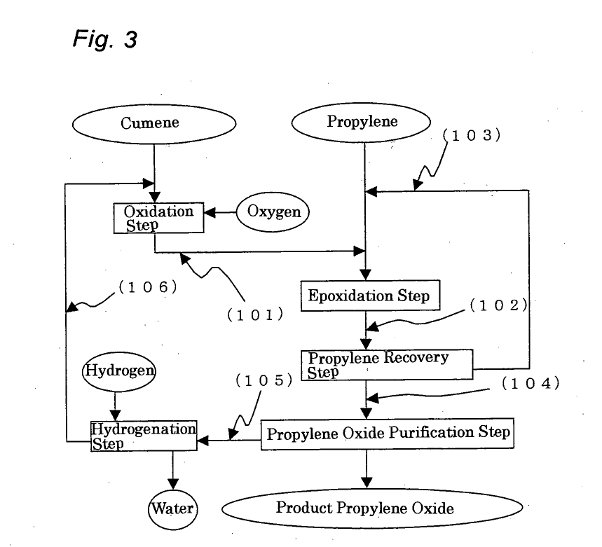

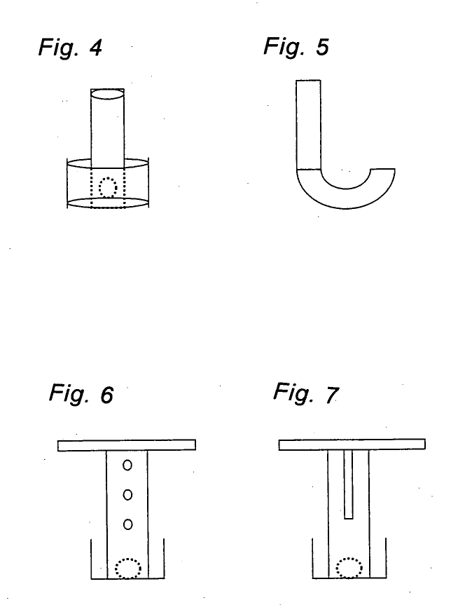

According to the method described in the specification as schematically shown in FIG. 3, using cumene as the alkylbenzene, an oxidation reaction liquid (101) containing 25% to 30% by weight of cumene hydroperoxide was obtained by oxidizing cumene with air in the oxidation step. An epoxidation reaction liquid (102) containing mainly propylene oxide, cumyl alcohol, unreacted propylene, and cumene was obtained by passing the oxidation reaction liquid and propylene through a reactor filled with a titanium-containing silicon oxide catalyst in the epoxidation step. Cumyl alcohol is an alcohol comes from the cumene hydroperoxide. The unreacted propylene (103) was separated and removed from the resulted reaction liquid (102) to obtain a reaction liquid (104) after recovering propylene. The reaction liquid (104) after recovering propylene was separated into a liquid fraction (105) containing mainly cumyl alcohol and cumene and a fraction containing mainly propylene oxide in the propylene oxide purification step, and then the fraction containing mainly propylene oxide was distilled with a plurality of distillation columns including extraction distillation so as to obtain a propylene oxide product which satisfies the product qualities. The liquid fraction (105) containing mainly cumyl alcohol and cumene as a continuous phase and a hydrogen gas as a dispersed phase was fed through the reactor in up-flow mode which was provided with i) a catalyst bed comprising an activated alumina catalyst and a palladium containing catalyst and ii) the gas-liquid dispersion device according to the present invention at the middle of the reactor, so that cumyl alcohol was reduced to cumene. The conversion of cumyl alcohol to cumene was more than 98%. The obtained cumene was recycled to the oxidation step. FIG. 3 illustrates the schematic flow-chart of Example 3 described in the specification.

### Comparative Experiment 3

The same procedure for the production of propylene oxide as in Example 3 was carried out except that the gas-liquid dispersion device according to the present invention was not used. An indication of channeling was observed, and the experimental results were not stable.

### INDUSTRIAL APPLICABILITY

The present invention provides the gas-liquid dispersion device and a method for producing the gas-liquid dispersion utilizing the device in the column wherein the gas-liquid mixed fluid flows upward which comprises the liquid as the continuous phase and the gas as the dispersed phase, characterized in that the gas is dispersed effectively into the liquid, thereby sufficient contact between the gas and the liquid can be attained, and furthermore the production of the device is comparatively easy. The present invention also provides an extremely effective method for producing the gas-liquid dispersion in the hydrogenation step in the propylene oxide production process.

1. A gas-liquid dispersion device comprising a plate for blocking a flow of a fluid and a conduit for conducting the fluid through the plate from a lower side to an upper side of the plate in a column wherein a gas-liquid mixed fluid flows upward which fluid comprises a liquid as a continuous phase and a gas as a dispersed phase, characterized in that:
(A) the plate has at least one hole through which the gas and the liquid pass,
(B) one end of the conduit is connected to the hole at a lower surface of the plate so that the conduit extends downward,
(C) at least one passage for the gas is provided through a side surface of the conduit,
(D) at least one passage for the liquid is provided in a lower part of the conduit, and
(E) an end of the lower part of the conduit has a structure for preventing the gas from flowing into the conduit.

2. The gas-liquid dispersion device according to claim 1, wherein the end of the lower part of the conduit has the structure which is closed by a cap.

3. The gas-liquid dispersion device according to claim 1, wherein the part of the lower part of the conduit has the structure which is bent in the form of “J” character.

4. The gas-liquid dispersion device according to claim 1, wherein a diameter of the hole of the plate is generally the same as that of the conduit.

5. The gas-liquid dispersion device according to claim 1, wherein the following equation (1) is satisfied:
1≦N/S≦100 (1)
wherein N is the number of the holes of the plate, and S is an area [m²] of the lower surface of the plate.

6. The gas-liquid dispersion device according to claim 1, wherein the following equation (2) is satisfied:
0.01≦v≦10 (2)
wherein v is a linear velocity [m/s] of the gas-liquid mixed fluid flowing through the hole of the plate.

7. The gas-liquid dispersion device according to claim 1, wherein the following equation (3) is satisfied:
100≦1.5×L≦H (3)
wherein H is a height of the column [mm], and L is a length of the conduit [mm].

8. The gas-liquid dispersion device according to claim 1, wherein the following equation (4) is satisfied:
10≦g≦500 (4)
wherein g is a velocity [m/s] of the gas flowing through the passage for the gas provided through the side surface of the conduit.

9. The gas-liquid dispersion device according to claim 1, wherein the following equation (5) is satisfied:
1≦h≦10 (5)
wherein h is a velocity [m/s] of the liquid flowing through the passage for the liquid provided through the conduit.

10. The gas-liquid dispersion device according to claim 1, wherein the following equation (6) is satisfied:
−4≦dP_{G}−dP_{L} (6)
wherein dP_{G} is the pressure drop across the passage for gas [kPa], and dP_{L} is the pressure drop across the passage for liquid [kPa].

11. The gas-liquid dispersion device according to claim 1, wherein two or more of the gas passages per one conduit are provided through the conduit and the passages for the gas are positioned at two or more different levels of the conduit.

12. The gas-liquid dispersion device according to claim 1, wherein at least one slit-type opening per one conduit is provided longitudinally through the side surface of the conduit, and at least a part of the opening functions as the passage for the gas.

13. The gas-liquid dispersion device according to claim 1, further comprising a bed of a packing above and/or below the gas-liquid dispersion device.

14. The gas-liquid dispersion device according to claim 13, wherein the packing comprises a catalyst.

15. The gas-liquid dispersion device according to claim 14, wherein the catalyst is a catalyst for hydrogenation reaction or dehydration reaction.

16. A method for producing a gas-liquid dispersion comprising a gas as a dispersed phase and a liquid as a continuous phase, comprising the steps of:
i) flowing a mixed fluid of the gas and the liquid upward through a column comprising the gas-liquid dispersion device according to claim 1;
ii) forming a space comprising the gas under the plate;
iii) conducting the liquid into the conduit through the passage for the liquid provided through the lower part of the conduit;
iv) conducting the gas into the conduit through the passage for the gas provided through the side surface of the conduit;
v) forming a gas-liquid mixed fluid by mixing the liquid and the gas in the conduit;
vi) then, flowing the gas-liquid mixed fluid upward through the hole for gas-liquid mixed fluid provided on the plate.

17. The method for producing the gas-liquid mixed dispersion of claim 15, characterised in that it is used in a hydrogenation step in the production of propylene oxide comprising the steps of:
i) an oxidation step for obtaining an aklylbenzene hydoroperoxide by oxidizing an alkylbenzene;
ii) an epoxidation step for obtaining a reaction liquid containing propylene oxide and an alcohol derived from the alkylbenzene hydroperoxide by reacting the alkylbenzene hydroperoxide and propylene in the presence of a catalyst;
iii) a propylene recovery step for recovering unreacted propylene from the reaction liquid after the epoxidation step, and for recycling the recovered propylene to the epoxidation step as a raw material;
iv) a propylene oxide purification step for obtaining a purified propylene oxide by, for example, distilling the propylene oxide obtained by the epoxidation step; and
v) a hydrogenation step for obtaining the alkylbenzene by hydrogenation, in the presence of a catayst, of the alcohol obtained from the alkylbenzene hydroperoxide via the epoxidation step for recycling the alkylbenzene as a raw material to the oxidation step.
